SEQUENCE LISTING (4 sequences)

SEQ ID NO: 1 - protein, 137 aa, Artificial Sequence
  modified INTEIN (Nostoc punctiforme)
GLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVERDHNFALKNGFIASN

SEQ ID NO: 2 - protein, 137 aa, Artificial Sequence
  modified INTEIN (Nostoc punctiforme)
ALSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVERDHNFALKNGFIASN

SEQ ID NO: 3 - DNA, 411 bp, Artificial Sequence
  Nucleic acid sequence for modified INTEIN
ggcctgagctatgaaaccgaaattctgaccgtggaatatggcctgctgccgattggtaaaattgtggaaaaacgcatcgaatgtactgtttatagcgttgataataatggcaatatttatacccagccggtggcacagtggcacgatcgcggcgaacaggaagtgtttgaatattgtctggaagatggtagcctgattcgcgcaaccaaagaccataaatttatgactgttgatggtcagatgctgccgattgatgaaatttttgaacgtgaactggatctgatgcgcgttgataatctgccgaatatcaaaattgcgacccgtaaatatctgggcaaacagaatgtgtatgacattggcgttgaacgcgaccataattttgcgctgaaaaatggcttcattgcttctaat

SEQ ID NO: 4 - DNA, 411 bp, Artificial Sequence
  Nucleic acid sequence for modified INTEIN
gcgctgagctatgaaaccgaaattctgaccgtggaatatggcctgctgccgattggtaaaattgtggaaaaacgcatcgaatgtactgtttatagcgttgataataatggcaatatttatacccagccggtggcacagtggcacgatcgcggcgaacaggaagtgtttgaatattgtctggaagatggtagcctgattcgcgcaaccaaagaccataaatttatgactgttgatggtcagatgctgccgattgatgaaatttttgaacgtgaactggatctgatgcgcgttgataatctgccgaatatcaaaattgcgacccgtaaatatctgggcaaacagaatgtgtatgacattggcgttgaacgcgaccataattttgcgctgaaaaatggcttcattgcttctaat

(19) United States
(12) Patent Application Publication
(10) Pub. No.: US 2020/0002743 A1
(43) Pub. Date: Jan. 2, 2020

(54) CONTROL SELF-CLEAVING PROTEIN ACTIVITY AND APPLICATION THEREOF

(71) Applicant: NATIONAL TSING HUA UNIVERSITY, Hsinchu (TW)

(72) Inventors: Yi-Zong LEE, Hsinchu (TW); Shih-Che SUE, Hsinchu (TW)

(21) Appl. No.: 16/116,120

(22) Filed: Aug. 29, 2018

(30) Foreign Application Priority Data
Jun. 29, 2018 (TW) 107122675

(51) Int. Cl. C12P 21/06; C12N 15/62; C12N 15/10; C07K 14/195

(52) U.S. Cl. CPC C12P 21/06; C12N 15/62; C12N 15/1065; C07K 2319/02; C07K 2319/21; C07K 2319/23; C07K 2319/50; C07K 14/195

(57) ABSTRACT

The invention provides a self-cleaving protein and a protein preparation method by employing a self-cleaving protein. The self-cleaving protein comprises an intein domain, wherein the intein domain contains at least one residue mutation at its N-terminus. The self-cleavage is prohibited under an “prohibition condition” and when the self-cleaving protein is under an “inducement condition”, the C-terminal cleavage will occur but N-terminal cleavage will not. In addition, the invention provides a fusion protein comprising the self-cleaving protein and target protein.

### CROSS-REFERENCE TO RELATED APPLICATIONS

This non-provisional application claims priority under 35 U.S.C. § 119 on Patent Application No. TW107122675 filed in Taiwan, Republic of China Jun. 29, 2018, the entire contents of which are hereby incorporated by reference in its entirety.

### FIELD OF THE INVENTION

The present invention relates to a self-cleaving protein, a fusion protein comprising the self-cleaving protein, and a method for producing target proteins by using the self-cleaving protein.

### BACKGROUND OF THE INVENTION

Inteins are naturally occurring, self-splicing protein domains that are capable of excising out their own protein domain from a larger protein structure while simultaneously joining the two formerly flanking peptide regions (“exteins”) together to form a mature host protein.

The ability of inteins to rearrange the flanking peptide bonds, and retain self-splicing activity when infusion with their native exteins, has led to a number of intein-based biotechnologies. These include various types of protein ligaton and activation applications, as well as protein labeling and detecting methods. An important application of inteins is in the production of recombinant proteins. In particular, inteins have the ability to impart self-cleaving activity to a number of conventional affinity and purification tags, and thus provide a major advance in the production of recombinant proteins for research, medical and other commercial applications.

Conventional fusion proteins and protein tags come with great advantage in producing recombinant proteins and polypeptides that a protein sequence is fused with the target protein to enhance expression and purification of the target protein. This method is already widely used. Although the advantage significantly contributes in protein production, however, the incorporation of the fused sequence in the final product creates drawbacks such as additional toxicity, target protein activity reduction, immune response and others. Therefore, to remove the fusion sequence is required for final product. The general strategy is to add protein enzyme to cleave the fused sequence from target protein. However, the cost of protein enzyme and further step to remove are not favorable for large-scale protein production.

Thus, incorporating intein self-cleaving activity to conventional protein tags create a significant advance, and early implementations of intein-based self-cleaving affinity tag systems have been published in several patents and many journal papers. However, several substantial weaknesses still remain that inhibit the full implementation of intein methods. For example, in order to be useful, the intein self-cleaving reaction must be tightly suppressed during protein expression and purification, but activated only when the tagged target protein is purified. Some conventional inteins are controlled by including thiol compounds (e.g., dithiothreitol) to trigger the auto-cleavage so that the extra cost is the processes for adding and removing thiol compound.

Accordingly, a stable and cost-effective method for purifying proteins, without enzymes or thiol compounds, is necessary.

### SUMMARY OF THE INVENTION

In view of the above-mentioned problem, the present invention provides a self-cleaving protein, a fusion protein containing the self-cleaving protein, and a method for producing target proteins using the self-cleaving protein.

The present invention provides a self-cleaving protein comprising an intein domain, wherein the intein domain comprises at least one residue mutation at its N-terminus to remove the N-terminal cleaving activity, but maintain the C-terminal cleaving activity that is able to be regulated by ionic strength, pH value, and temperature.

In one embodiment, the mutation at N-terminus comprises the substitution, deletion, insertion, and/or replacement of amino acid residues.

In one embodiment, the intein domain is derived from NpuDnaE.

In one embodiment, the first amino acid residue of the intein domain is changed to other amino acids from Cys, but not changed to Ser, Thr.

In one embodiment, the self-cleaving protein has an amino acid sequence selected from the group consisting of SEQ ID NO: 1 and 2.

In one embodiment, the self-cleaving protein has an amino acid sequence of

(SEQ ID NO: 1)
GLSYETEILTVEYGLLPIGKIVEKRIECTVYSVDNNGNIYTQPVAQWH
DRGEQEVFEYCLEDGSLIRATKDHKFMTVDGQMLPIDEIFERELDLMR
VDNLPNIKIATRKYLGKQNVYDIGVERDHNFALKNGFIASN.

In one embodiment, the self-cleaving protein has an amino acid sequence of

(SEQ ID NO: 2)
ALSYETEILTVEYGLLPIGKIVEKRIECTVYSVDNNGNIYTQPVAQWH
DRGEQEVFEYCLEDGSLIRATKDHKFMTVDGQMLPIDEIFERELDLMR
VDNLPNIKIATRKYLGKQNVYDIGVERDHNFALKNGFIASN.

The present invention also provides a fusion protein having a structure of the following formula (I):

(X)n-(I)-(P)m (I)
wherein
“X” is an amino acid sequence, linker, magnetic beads, or the like;
“I” is a self-cleaving protein of the present invention;
“P” is a target protein;
“n” may be an integer greater than 0;
“m” may be an integer greater than 1.

In one embodiment, the “X” is a peptide tag comprising His-Tag, GST-Tag, or other amino acid sequences that can be useful for protein purification or expression. Preferably, the tag is used to collect and purify the fusion proteins under a neutral or acidic pH condition.

In one embodiment, the target protein “P” comprises polypeptides, proteins, recombinant proteins, antibodies, enzymes, or hormones.

The present invention further provides a protein expression vector comprising the nucleic acid sequences of the self-cleaving protein.

In one embodiment, the protein expression vector comprises a nucleic acid sequences encoding His-Tag, GST-Tag, or other amino acid sequences that can be useful for protein purification or expression. Preferably, the tag is used to collect and purify the fusion proteins at a neutral or acidic pH condition.

In one embodiment, the self-cleaving protein has a nucleic acid sequence selected from the group consisting of SEQ ID NO: 3 and 4.

In one embodiment, the self-cleaving protein has a nucleic acid sequence of

(SEQ ID NO: 3)
GGCCTGAGCTATGAAACCGAAATTCTGACCGTGGAATATGGCCTGCTG
CCGATTGGTAAAATTGTGGAAAAACGCATCGAATGTACTGTTTATAGC
GTTGATAATAATGGCAATATTTATACCCAGCCGGTGGCACAGTGGCAC
GATCGCGGCGAACAGGAAGTGTTTGAATATTGTCTGGAAGATGGTAGC
CTGATTCGCGCAACCAAAGACCATAAATTTATGACTGTTGATGGTCAG
ATGCTGCCGATTGATGAAATTTTTGAACGTGAACTGGATCTGATGCGC
GTTGATAATCTGCCGAATATCAAAATTGCGACCCGTAAATATCTGGGC
AAACAGAATGTGTATGACATTGGCGTTGAACGCGACCATAATTTTGCG
CTGAAAAATGGCTTCATTGCTTCTAAT.

In one embodiment, the self-cleaving protein has a nucleic acid sequence of

(SEQ ID NO: 4)
GCGCTGAGCTATGAAACCGAAATTCTGACCGTGGAATATGGCCTGCTG
CCGATTGGTAAAATTGTGGAAAAACGCATCGAATGTACTGTTTATAGC
GTTGATAATAATGGCAATATTTATACCCAGCCGGTGGCACAGTGGCAC
GATCGCGGCGAACAGGAAGTGTTTGAATATTGTCTGGAAGATGGTAGC
CTGATTCGCGCAACCAAAGACCATAAATTTATGACTGTTGATGGTCAG
ATGCTGCCGATTGATGAAATTTTTGAACGTGAACTGGATCTGATGCGC
GTTGATAATCTGCCGAATATCAAAATTGCGACCCGTAAATATCTGGGC
AAACAGAATGTGTATGACATTGGCGTTGAACGCGACCATAATTTTGCG
CTGAAAAATGGCTTCATTGCTTCTAAT.

The present invention further provides a method for producing and purifying proteins, comprising:
(a) expressing a fusion protein in a host cell;
(b) collecting and purifying the fusion protein under a prohibition condition;
(c) separating a target protein from a self-cleaving protein under an inducement condition when the C-terminal cleavage occurs; and
(d) collecting and purifying the target protein.

In one embodiment, the prohibition condition is a neutral or acidic pH condition.

In one embodiment, the prohibition condition is a high ionic strength condition.

In one embodiment, the prohibition condition is a condition of pH 4-7, more than 300 mM salt, and/or lower than 25° C.

In one embodiment, the inducement condition is an alkaline pH condition.

In one embodiment, the inducement condition is a low ionic strength condition.

In one embodiment, the inducement condition is a condition of pH 7-11, salt concentration less than 300 mM, and/or temperature higher than 25° C.

In one embodiment, the salt comprises NaCl and other charged water-soluble ions.

In order to achieve the purpose stated above, as well as other purposes, the characteristics and advantages of the present invention can be more apparent and understandable, and preferred embodiments are exemplified in the following with accompanying drawings to aid in detailed interpretation.

### BRIEF DESCRIPTION OF THE DRAWINGS

FIG. 1 is a schematic diagram showing the structure of the fusion protein of the present invention. FIG. 1 shows that the fusion protein is cleaved under a specific condition.

FIG. 2 illustrates an E. coli plasmid map of pET-NpuDnaE_{C1G} used in Examples.

FIG. 3 illustrates an SDS-PAGE profile of NpuDnaE_{C1G}-Mms6 fusion protein produced by methods disclosed herein, wherein the Mms6 is a target polypeptide. FIG. 3 shows the SDS-PAGE profile of samples from the different stages of the expression and purification of the NpuDnaE_{C1G}-Mms6 fusion protein.

FIG. 4 illustrates an SDS-PAGE profile of NpuDnaE_{C1G}-Mms6 fusion protein before and after cleavage. The NpuDnaE_{C1G}-Mms6 fusion protein is cleaved into two protein fragments after cleavage.

FIG. 5 illustrates an MALDI-TOF MS profile of Mms6. The molecular weight of the Mms6 protein, separated from NpuDnaE_{C1G}-Mms6 fusion protein, has the same value as theoretical molecular weight.

### DETAILED DESCRIPTION OF THE INVENTION

A self-cleaving fusion protein is provided. The self-cleaving fusion protein of the present invention comprises an intein domain, wherein the intein domain contains at least one mutation at its N-terminus to remove its N-terminal self-cleaving activity, but maintains the activity of C-terminal cleavage that is able to be regulated by ionic strength, pH value, and temperature.

The N-terminus of the intein domain having at least one mutation is a “modified intein” or “mutated intein”. The term “modified intein” or “mutated intein” refers to one or more modifications in amino acid sequence being referred to native intein. Such modification can be a substitution, addition, or deletion.

The amino acid substitution, addition, and insertion can be accomplished with natural or non-natural amino acids. Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. Non-naturally occurring amino acids include, but are not limited to, ε-N Lysine, ß-alanine, ornithine, norleucine, norvaline, hydroxyproline, thyroxine, γ-amino butyric acid, homoserine, citrulline, aminobenzoic acid, 6-Aminocaproic acid (Aca; 6-Aminohexanoic acid), hydroxyproline, mercaptopropionic acid (MPA), 3-nitro-tyrosine, pyroglutamic acid, and the like.

In one embodiment, the self-cleaving protein of the present invention is derived from NpuDnaE. Its first amino acid residue at N-terminus is changed from cysteine (Cys) to other amino acid residues, preferably glycine (Gly) or alanine (Ala), but not Serine (Ser) or threonine (Thr). The mutated intein therefore doesn't have the N-terminal cleaving activity, but still retain the C-terminal cleaving activity. The self-cleaving protein of the present invention has an amino acid sequence selected from the group consisting of SEQ ID NO: 1 and 2.

In one embodiment, the N-terminal and C-terminal cleavage of the self-cleaving protein of the present invention are both inhibited under the “prohibition condition”. The “prohibition condition” includes a neutral or acidic pH condition, preferably, pH 4 to 7; a high ionic strength environment, preferably more than 300 mM NaCl; and a temperature lower than 25° C.

In one embodiment, the C-terminal cleavage of the self-cleaving protein of present invention is activated under the “inducement condition”. Because the N-terminal residue at of the self-cleaving protein of present invention is mutated or modified, only C-terminal self-cleavage still occurs.

The “inducement condition” includes an alkaline pH condition, preferably, pH 7 to 11; a low ionic strength environment, preferably less than 300 mM NaCl; and a temperature higher than 25° C.

The “salt” as used herein refers to NaCl and any other charged water-soluble ions.

The present invention also provides a fusion protein. The fusion protein comprises the self-cleaving protein of the present invention; a tag, linker, magnetic bead, or the like linked to the N-terminus of the self-cleaving protein; and a target protein linked to the C-terminus of the self-cleaving protein.

As used herein, the term “tag” refers to a peptide enabling a specific interaction with an affinity chromatography. For the purposes of affinity purification, the protein of interest is often “tagged” with a protein sequence that an affinity chromatography could bind. Several molecular tagging systems have been developed and used to generate fusion proteins incorporating tags including the following: myc tag; Flag-peptide tag; His-Tag; Strep-Tag; GST-Tag; MBP-Tag; SNAP-Tag; Halo-Tag; Tap-Tag; or INPACT-CN. Preferably, the tag contributes to collect and purify the fusion protein at neutral or acidic pH condition.

The “target protein” as used herein refers to a biological molecule, such as proteins or recombinant polypeptides.

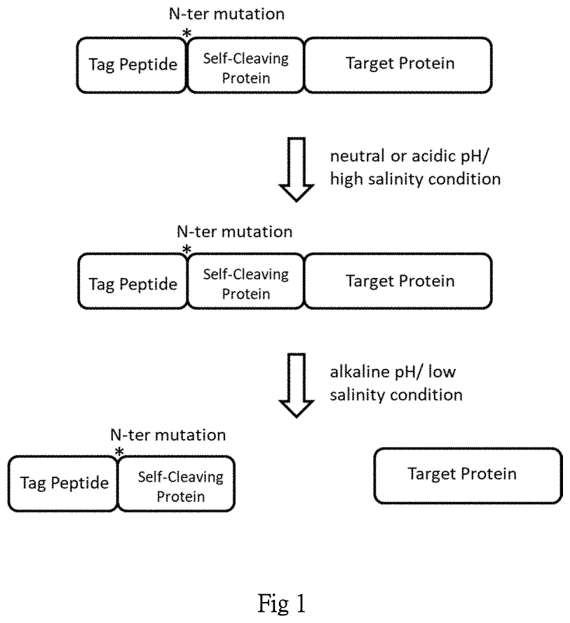

As described above, the self-cleaving protein of the present invention contains one or more mutations to inhibit its N-terminal cleaving activity. The self-cleaving protein and target protein are able to be cleaved or remain linked under specific conditions. As shown in FIG. 1, a fusion protein includes a tag, a self-cleaving protein, and a target protein. The fusion protein expressed in host cells is not self-cleaved under the prohibition condition (such as neutral or acidic pH and high ionic strength environment). In contrast, the C-terminal cleavage will occur when the fusion protein is under the inducement condition so that the target protein is separated from the self-cleaving protein. The target protein can be obtained by well-known methods of protein isolation and purification.

The present invention also provides a method for producing and manufacturing a self-cleaving protein comprising: (a) expressing a fusion protein of the present invention in a host cell, (b) collecting and purifying the fusion protein under the prohibition condition, (c) separating a target protein from a self-cleaving protein under an inducement condition when the C-terminal cleavage occurs; and (d) collection and purification of the target protein.

An expression vector encoding a fusion protein can be prepared using techniques known in the field, and then introduced into a host cell for expressing the fusion protein. The fusion protein of the present invention can be produced in mammalian cells, lower eukaryotes, or prokaryotes. Examples of mammalian cells include monkey COS cells, CHO cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. The lower eukaryotes include yeast, such as Saccharomyces cerevisiae, Schizosaccharomyces pombe, Kluyveromyces strains, Candida, or any others. Prokaryotes include Escherichia coli, Bacillus subtilis, Salmonella typhimurium, or any others.

The present invention further provides an expression vector including a nucleic acid sequence encoding the self-cleaving protein of the present invention.

The “vector” as used herein includes, but is not limited to, pET, pQE, pICZa, pFastBac, pGFP2, or any other vectors that are capable to express heterogeneous proteins.

In one embodiment, the expression vector comprises the sequences of SEQ ID NO: 3 and 4.

In one embodiment, the vector comprises a nucleic acid sequence that encodes His-Tag, GST-Tag, or any other commercial tags. Preferably, the tag can be easily collected and purified at the neutral to acidic pH.

The fusion protein is preferred to be expressed, collected, and purified in an environment that is suitable for cell growth and doesn't cause protein cleavage. In one embodiment, the fusion can be expressed, collected, and purified under the prohibition condition.

Then, the fusion protein is treated under the inducement condition to allow the self-cleavage occurs between the target protein and self-cleaving protein (FIG. 1). Finally, the target protein is collected and purified.

The methods of the present invention provide significant advantages including: (1) a target protein can be cost-effectively obtained by simply controlling the solvent condition, without any requirement of enzymes or chemicals; (2) the self-cleaving protein (intein) has a high tolerance for downstream sequences; and (3) no residual residue remained on the target protein.

### Example 1

Preparation of Fusion Protein

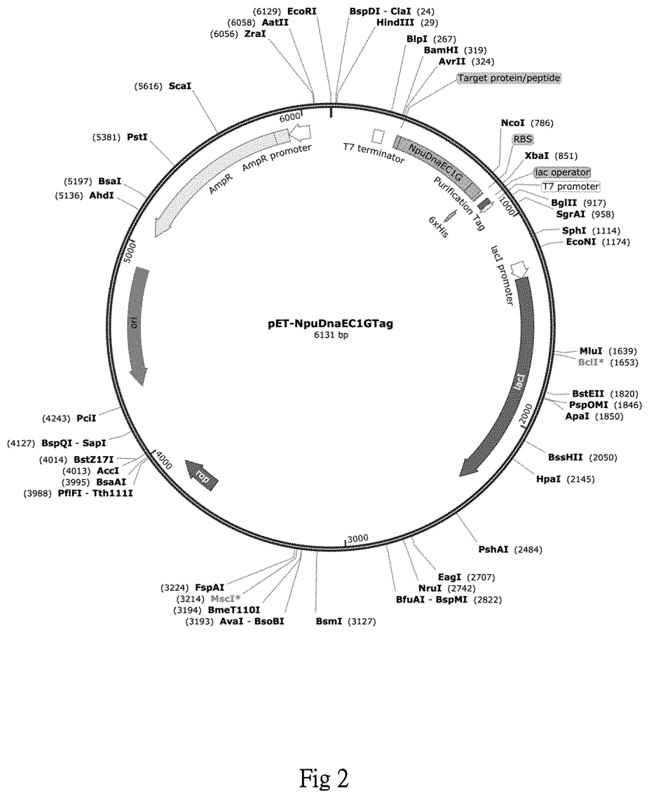

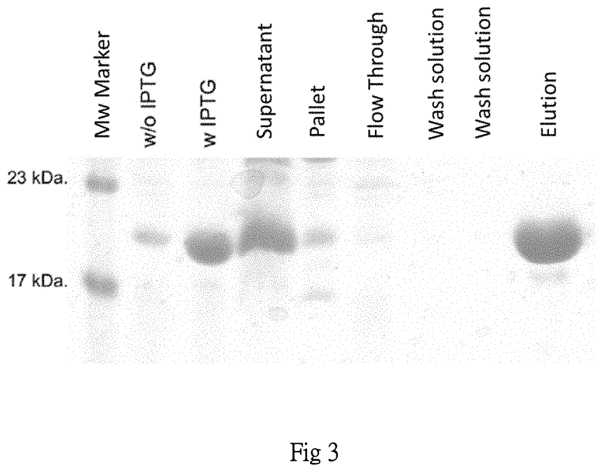

A expression vector pET-NpuDnaE_{C1G}tag (FIG. 2) including a nucleic acid encoding NpuDnaE_{C1G} fusion protein (containing His-Tag, NpuDnaE_{C1G}, and Mms6 polypeptide) was introduced into E. coli to express NpuDnaE_{C1G} fusion protein by IPTG induction. The NpuDnaE_{C1G} fusion protein was purified by nickel-charged resins under a high ionic strength environment (300 mM NaCl) for sodium dodecyl sulfate polyacrylamide gel electrophoresis (SDS-PAGE) analysis (FIG. 3).

According to FIG. 3, the NpuDnaE_{C1G} fusion protein was highly expressed in E. coli after IPTG induction. Most of the NpuDnaE_{C1G} fusion protein was in supernatant and purified by nickel-charged resins. The NpuDnaE_{C1G} fusion protein was not cleaved under high ionic strength environment (more than 300 mM NaCl).

### Example 2

Cleavage of Fusion Protein

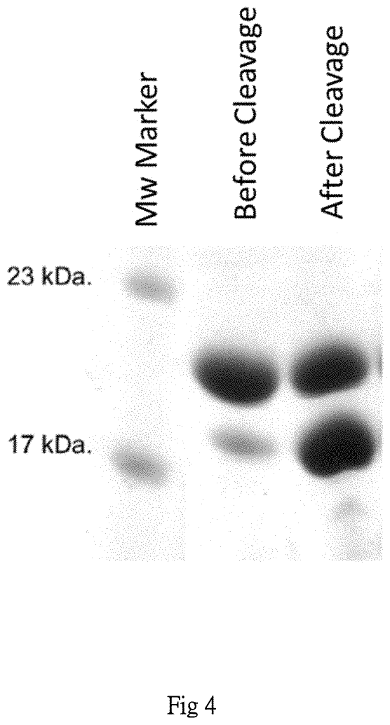

The salinity of the NpuDnaE_{C1G} fusion protein solution was reduced to less than 300 mM and the pH value of the fusion protein solution was increased to pH 10 by buffer exchange. After buffer exchange, the NpuDnaE_{C1G} fusion protein solution was stood at 50° C. for 30 minutes. The NpuDnaE_{C1G} fusion protein was separated using SDS-PAGE (FIG. 3). As shown in FIG. 4, the NpuDnaE_{C1G} fusion protein was cleaved into two fragments. One was 20-kDa fusion protein including His-tag, intein and Mms6 protein. Another was 17-kDa fusion protein only including His-tag and intein.

### Example 3

Mass Spectrometry Analysis

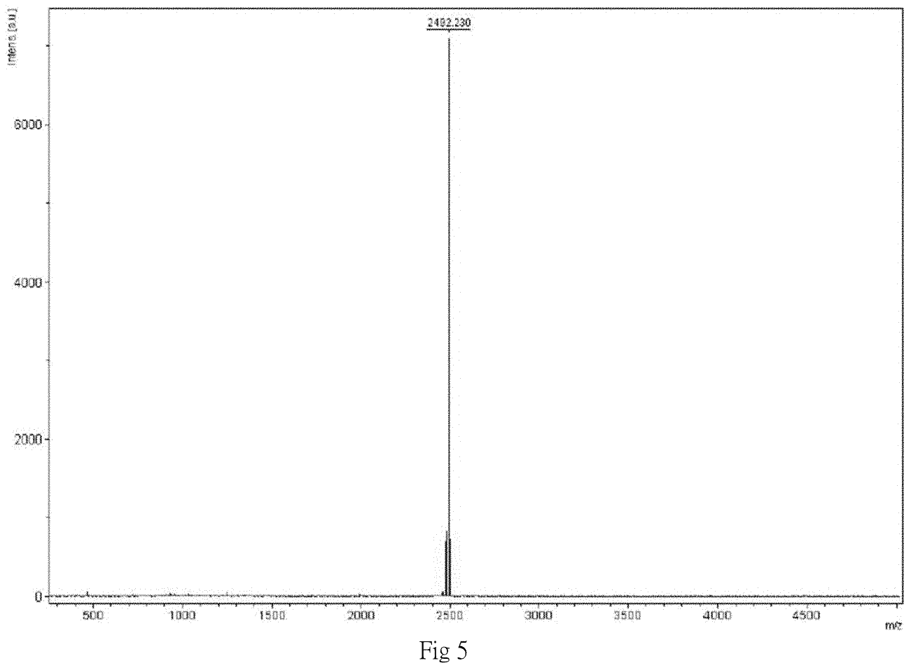

The intein (self-cleaving protein) and Mms6 (target protein) were separated by high performance liquid chromatography (HPLC), and then analyzed by MALDI-TOF MS to determine the correct molecule weight of the Mms6 protein (FIG. 5). As shown in FIG. 5, the molecule weight of Mms6 protein determined by MALDI-TOF MS was 2492.23 kDa that is almost identical to the theoretical value of Mms6 (2492.7 kDa).

What is claimed is:

1. A self-cleaving protein comprising an intein domain, wherein the intein domain comprises a mutation at position 1 of its N-terminus, and the intein domain is derived from NpuDnaE.

2. The self-cleaving protein according to claim 1, wherein the mutation comprises substitution, deletion, insertion, and/or replacement.

3. The self-cleaving protein according to claim 1, wherein the Cys residue at position 1 of the N-terminus of the intein is mutated to remove the N-terminal self-cleaving activity of the intein.

4. The self-cleaving protein according to claim 1, wherein the self-cleaving protein has an amino acid sequence selected from the group consisting of SEQ ID NO: 1 and SEQ ID NO:2.

5. A fusion protein having a structure of the following formula (I)
(X)n-(I)-(P)m (I)
wherein
“X” is an amino acid sequence, linker, magnetic beads, or the like;
“I” is a self-cleaving protein of claim 1;
“P” is a target protein;
“n” may be an integer greater than 0; and
“m” may be an integer greater than 1.

6. The fusion protein according to claim 5, wherein the “X” is a peptide tag that comprises any amino acid sequences that can be useful for protein purification or expression.

7. The fusion protein according to claim 6, wherein the tag peptide comprises His-Tag, GST-Tag or others.

8. The fusion protein according to claim 5, wherein the target protein comprises polypeptides, proteins, recombinant proteins, antibodies, enzymes, or hormones.

9. A protein expression vector comprising a nucleic acid sequence encoding the self-cleaving protein of claim 1.

10. The protein expression vector according to claim 9, wherein the nucleic acid sequence is selected from a group consisting of SEQ ID NO: 3 and SEQ ID NO: 4.

11. The protein expression vector according to claim 9, comprising a nucleic acid sequence encoding His-Tag, GST-Tag or others.

12. A method for producing and purifying protein comprising:
(a) expressing the fusion protein or claim 5 in a host cell;
(b) collecting and purifying the fusion protein under a prohibition condition, wherein the prohibition condition is a neutral to acidic pH condition;
(c) separating a target protein from the self-cleaving protein under an inducement condition, wherein the inducement condition is an alkaline pH condition; and
(d) collecting and purifying the target protein.

13. The method according to claim 12, wherein the neutral to acidic pH condition is pH 4-7.

14. The method according to claim 12, wherein the prohibition condition is a high ionic strength condition.

15. The method according to claim 14, wherein the ionic strength salinity condition has a salt concentration of more than 300 mM.

16. The method according to claim 12, wherein the prohibition condition has a temperature lower than 25° C.

17. The method according to claim 12, wherein the alkaline pH condition is pH 7-11.

18. The method according to claim 12, wherein the inducement condition is a low ionic strength condition.

19. The method according to claim 18, wherein the low ionic strength condition has a salt concentration of less than 300 mM.

20. The method according to claim 12, wherein the inducement condition has a temperature higher than 25° C.
